(19) United States
(12) Patent Application Publication
(10) Pub. No.: US 2024/0376549 A1
(43) Pub. Date: Nov. 14, 2024

(54) METHOD FOR DETECTING GENE MUTATION AND METHOD FOR DIFFERENTIATING SOMATIC CELL MUTATION FROM GERM CELL LINE MUTATION

(71) Applicants: SHIZUOKA PREFECTURE, Shizuoka (JP); SRL & SHIZUOKA CANCER CENTER COLLABORATIVE LABORATORIES INC., Shizuoka (JP)

(72) Inventors: Keiichi HATAKEYAMA, Shizouka (JP); Takashi SUGINO, Shizuoka (JP); Koji MURAMATSU, Shizuoka (JP); Keiichi OHSHIMA, Shizuoka (JP); Hirotsugu KENMOTSU, Shizuoka (JP); Kenichi URAKAMI, Shizuoka (JP); Yuji SHIMODA, Shizuoka (JP); Takeshi NAGASHIMA, Tokyo (JP); Yuichi KAWANISHI, Shizuoka (JP); Ryutaro FUKUMURA, Shizuoka (JP)

(21) Appl. No.: 18/685,363

(22) PCT Filed: Aug. 23, 2022
(86) PCT No.: PCT/JP2022/031772

§ 371 Date: Feb. 21, 2024

(30) Foreign Application Priority Data
Aug. 23, 2021 (JP) 2021135550

(51) Int. Cl. C12Q 1/6886; C12Q 1/6806; C12Q 1/6874

(52) U.S. Cl. CPC C12Q 1/6886; C12Q 1/6874; C12Q 1/6806; C12Q 2600/156; C12Q 2600/158

(57) ABSTRACT

Provided are a method for detecting a gene mutation using an FFPE tissue section containing tumor cells regardless of the percentage of tumor cells, the method being capable of increasing the number of detectable gene mutations and mutant allele frequency, and a method capable of differentiating, even in the absence of blood samples, a somatic cell mutation from a germ cell line mutation. A method for detecting a gene mutation according to the present invention comprises: a dissociation step for dissociating a single cell population from a formalin-fixed paraffin-embedded tissue section containing tumor cells; a separation step for obtaining a tumor fraction containing the tumor cells from the single cell population; a collection step for collecting a nucleic acid molecule from the tumor fraction; and a sequencing step for subjecting the nucleic acid molecule to sequencing.

### TECHNICAL FIELD

The present invention relates to a method for detecting a gene alteration and a method for distinguishing between a somatic mutation and a germline mutation.

### BACKGROUND ART

In the treatment of cancer, limited genetic testing, such as companion diagnostics, provides cancer patients and clinicians with important information for effective selection of drugs. Recent large-scale analyses using next-generation sequencing (hereinafter also referred to as “NGS”) have revealed the relationship between gene alterations and various cancers (Non-Patent Documents 1 to 3). Based on these findings, sequencing of multiple target gene panels using NGS provides an opportunity for further drug selection in clinical practice. Citation List Patent Document
Non-Patent Document 1: Alexandrov, L. B. et al., Nature, 2013, Vol. 500, pp. 415-421
Non-Patent Document 2: Consortium, I. T. P.-C. A. o. W. G., Nature, 2020, Vol. 578, pp. 82-93
Non-Patent Document 3: Nagashima, T. et al., Cancer Sci, 2020, Vol. 111, pp. 687-699

### DISCLOSURE OF THE INVENTION

### Problems to be Solved by the Invention

The detection of somatic mutations using NGS is affected by the tumor content in tissue samples. Generally, sequencing of a target panel is performed using formalin-fixed, paraffin-embedded (hereinafter also referred to as “FFPE”) tissue sections. Such FFPE tissue sections with low tumor content can be subjected to tumor cell enrichment by macrodissection. However, for cancers, such as diffuse-type gastric cancer or lobular breast cancer, macrodissection is often unsuitable because of the diffused type of tumor cells. In many cases, especially in the diffuse-type gastric cancer, the estimated content of tumor cells is 30% or less. Therefore, alternative tumor cell enrichment methods besides macrodissection are required for accurate detection of mutations in the sequencing of a target panel of genes for various cancer types.

The targeted sequencing has two standard pipelines for detection of somatic mutations, one using blood as a reference and the other using public databases. Although the pipeline using the databases has the advantage that FFPE tissue sections can be analyzed without the need for a blood reference, this approach entails the risk that alterations derived from germline mutations are falsely detected as derived from somatic mutations. In other words, the accuracy of detection of somatic mutations depends on public databases owing to population stratification in single nucleotide polymorphisms (SNPs), because of which false positive mutations are increased for populations with insufficient SNP information. In contrast, in the pipeline using blood from the same patient from whom tissue is obtained, germline mutations can be reliably determined by subtracting mutations detected in a blood reference, resulting in the extraction of only somatic mutations upon targeted sequencing. However, most archived specimens stored as FFPE tissue sections are not paired with a blood reference that could allow detection of somatic mutations based on targeted sequencing.

The present invention is made in view of the problem mentioned above, and an object thereof is to provide a method for detecting a gene alteration that enables improvement in a number of detectable gene alterations and a variant allele frequency using an FFPE tissue section including a tumor cell regardless of a proportion of the tumor cell and a method for distinguishing between a somatic mutation and a germline mutation without a blood sample.

### Means for Solving the Problems

The present inventors conducted extensive studies to solve the above problem. As a result, the present inventors have found that the above problem can be solved by dissociating a single cell population from an FFPE tissue section including a tumor cell and obtaining a tumor fraction including the tumor cell from the single cell population to thereby enrich the tumor cell. Thus, the present invention has completed. More specifically, the present invention can provide the following.

(1) A method for detecting a gene alteration, the method including:
dissociating a single cell population from a formalin-fixed, paraffin-embedded tissue section including a tumor cell;
separating a tumor fraction including the tumor cell from the single cell population;
collecting a nucleic acid molecule from the tumor fraction; and sequencing the nucleic acid molecule.

(2) The method for detecting a gene alteration according to (1), in which the formalin-fixed, paraffin-embedded tissue section has a thickness of 10 μm or more and 50 μm or less.

(3) The method for detecting a gene alteration according to (1) or (2), in which the nucleic acid molecule is DNA.

(4) The method for detecting a gene alteration according to any one of (1) to (3), in which the sequencing is next-generation sequencing.

(5) The method for detecting a gene alteration according to any one of (1) to (4), in which the separating includes binding the tumor cell to a magnetic bead and separating, from cells other than the tumor cell by an action of magnetism, the magnetic bead to which the tumor cell has bound,
the magnetic bead having a ligand that specifically binds to a biomolecule specifically present in the tumor cell.

(6) The method for detecting a gene alteration according to (5), in which the biomolecule is at least one selected from the group consisting of cytokeratin and gene products of the below-described genes and the ligand is an antibody against the biomolecule:
a HJURP gene, a KIF2C gene, a ASPN gene, a GINS1 gene, a NUSAP1 gene, a IQGAP3 gene, a CDK1 gene, a TPX2 gene, a CDT1 gene, a MMP11 gene, a MEX3A gene, a TUBB3 gene, a BIRC5 gene, a HIST2H3A gene, a CENPF gene, a CCNB2 gene, a TROAP gene, a CDCA5 gene, a KIAA0101 gene, a UBE2C gene, a AURKB gene, a CKAP2L gene, a CEP55 gene, a EXO1 gene, a KIF20A gene, a CCNA2 gene, a HIST1H2AL gene, a ANLN gene, a CENPA gene, a TTK gene, a ORC6 gene, a SHCBP1 gene, a FOXM1 gene, a MELK gene, a SPC25 gene, a TOP2A gene, a BUB1B gene, a MAD2L1 gene, a MND1 gene, a KIFC1 gene, a NUF2 gene, a GTSE1 gene, a E2F1 gene, a BUB1 gene, a DLGAP5 gene, and a KIF14 gene.

(7) The method for detecting a gene alteration according to (5) or (6), in which the biomolecule is cytokeratin and the ligand is an anti-cytokeratin antibody.

(8) A method for distinguishing between a somatic mutation and a germline mutation, the method including:
the dissociating, the separating, the collecting, and the sequencing in the method for detecting a gene alteration according to any one of (1) to (7), and
further including:
secondarily collecting a nucleic acid molecule from a residual fraction remaining after obtaining the tumor fraction in the separating;
secondarily sequencing the nucleic acid molecule collected in the secondarily collecting; and
estimating, for a target mutation detected in the sequencing, whether the target mutation is a germline mutation or not based on at least one of a variant allele frequency obtained in the sequencing and a variant allele frequency obtained in the secondarily sequencing.

### Effects of the Invention

The present invention can provide a method for detecting a gene alteration that enables improvement in a number of detectable gene alterations and a variant allele frequency using an FFPE tissue section including a tumor cell regardless of a proportion of the tumor cell and a method for distinguishing between a somatic mutation and a germline mutation without a blood sample.

### BRIEF DESCRIPTION OF THE DRAWINGS

FIG. 1 represents optical micrographs showing diffuse-type gastric cancers (D1 and D2) and intestinal gastric cancers (S1 and S2) used in Example. FFPE tissue sections stained with Hematoxylin and eosin were used. Scale bar represents 2.5 mm. In insets of the micrographs, areas with a high density of tumor cells are indicated with black arrows. Scale bar represents 100 μm.

FIG. 2 represents graphs showing amounts of tumor cells in unseparated samples, tumor fractions, and residual fractions obtained in Example.

FIGS. 3A to 3D represent graphs showing quality of DNA extracted from unseparated samples, tumor fractions, and residual fractions obtained in Example. FIG. 3A represents graphs showing a DNA concentration. FIG. 3B represents graphs showing a DNA integrity number (DIN). FIG. 3C represents graphs showing an average of read depth. FIG. 3D represents graphs showing an estimated tumor content.

FIGS. 4A to 4E represent graphs showing an influence of tumor cell enrichment on detection of a somatic mutation. FIG. 4A represents a graph showing a number of nonsynonymous mutations. FIG. 4B represents a Venn diagram showing distribution of nonsynonymous mutations among an unseparated sample, a tumor fraction, and a residual fraction. FIG. 4C represents graphs showing a variant allele frequency (VAF) (left) and read depth (right). * represents p<0.01/3 (Welch's t-test with Bonferroni correction). FIG. 4D represents a graph showing a frequency of somatic mutations detected in diffuse-type and intestinal gastric cancers. FIG. 4E represents a graph showing variations in VAF in an unseparated sample, a tumor fraction, and a residual fraction.

FIGS. 5A to 5C represent graphs showing characteristics of somatic and germline mutations in an unseparated sample, a tumor fraction, and a residual fraction. FIG. 5A represents graphs showing distribution of VAF (left) and read depth (right). * represents p<0.01. FIG. 5B represents a graph showing a ratio of VAF in mutations shared in an unseparated sample, a tumor fraction, and a residual fraction ((c) in FIG. 4B) as compared between germline mutation and somatic mutation. * represents p<0.01. FIG. 5C represents a graph showing a receiver operating characteristic (ROC) curve for estimation of germline and somatic mutations.

FIG. 6 represents a diagram showing a heat map obtained by clustering expression levels in a tumor site plotted with 21 tumor types and 46 genes used in Example as axes.

FIG. 7 represents a diagram showing a frequency and intracellular localization of expression of 46 genes used in Example in tumor and normal tissues.

### PREFERRED MODE FOR CARRYING OUT THE INVENTION

### <Method for Detecting Gene Alteration>

A method for detecting a gene alteration according to the present invention includes
dissociating a single cell population from an FFPE tissue section including a tumor cell;
separating a tumor fraction including the tumor cell from the single cell population;
collecting a nucleic acid molecule from the tumor fraction; and
sequencing the nucleic acid molecule. The method for detecting a gene alteration according to the present invention can improve a number of detectable gene alterations and a variant allele frequency using an FFPE tissue section including a tumor cell regardless of a proportion of the tumor cell.

### [Dissociation Step]

In a dissociation step, a single cell population is dissociated from an FFPE tissue section including a tumor cell. A method for dissociating is not particularly limited and known methods may be used.

A thickness of the FFPE tissue section is not particularly limited and, for example, may be 10 μm or more and 50 μm or less, preferably 10 μm or more and 20 μm or less from the viewpoints of resource saving and consistency with conventional methods, and more preferably 10 μm.

A proportion of the tumor cell in the FFPE tissue section is not particularly limited. The method for detecting a gene alteration according to the present invention can improve a number of detectable gene alterations and a variant allele frequency even when the proportion is low, for example, 30% or less and preferably 15 to 25%. Note that, the proportion is measured as a proportion of an area occupied by tumor cells in the FFPE tissue section to an area occupied by the FFPE tissue section in an optical micrograph of the FFPE tissue section. The FFPE tissue section may be, for example, stained with Hematoxylin and eosin.

### [Separation Step]

In a separation step, a tumor fraction including the tumor cell is obtained from the single cell population. At that time, a tumor fraction including the tumor cell may be obtained by separating the tumor cell from the single cell population and collecting the thus-separated tumor cell, or by separating cells other than the tumor cell from the single cell population and then collecting a remainder.

A method for separating the tumor cell is not particularly limited and known methods may be used. The method for separating may be, for example, a method using a biomolecule specifically present in the tumor cell. Specifically, for example, the tumor cell is bound to a ligand that specifically binds to the biomolecule via the biomolecule and the ligand to which the tumor cell has bound is collected. The above-described biomolecule may be used alone or two or more thereof may be used in combination. The above-described ligand may be used alone or two or more thereof may be used in combination.

In one embodiment, the biomolecule may be, for example, at least one selected from the group consisting of cytokeratin and gene products of the below-described genes. The gene products may be, for example, proteins. The ligand may be, for example, an antibody against the biomolecule.

a HJURP gene, a KIF2C gene, a ASPN gene, a GINS1 gene, a NUSAP1 gene, a IQGAP3 gene, a CDK1 gene, a TPX2 gene, a CDT1 gene, a MMP11 gene, a MEX3A gene, a TUBB3 gene, a BIRC5 gene, a HIST2H3A gene, a CENPF gene, a CCNB2 gene, a TROAP gene, a CDCA5 gene, a KIAA0101 gene, a UBE2C gene, a AURKB gene, a CKAP2L gene, a CEP55 gene, a EXO1 gene, a KIF20A gene, a CCNA2 gene, a HIST1H2AL gene, a ANLN gene, a CENPA gene, a TTK gene, a ORC6 gene, a SHCBP1 gene, a FOXM1 gene, a MELK gene, a SPC25 gene, a TOP2A gene, a BUB1B gene, a MAD2L1 gene, a MND1 gene, a KIFC1 gene, a NUF2 gene, a GTSE1 gene, a E2F1 gene, a BUB1 gene, a DLGAP5 gene, and a KIF14 gene

In another embodiment, the biomolecule may be, for example, a protein specifically present in the tumor cell such as cytokeratin and EpCAM. The ligand may be, for example, an antibody against the protein.

A method for separating the cells other than the tumor cell is not particularly limited and known methods may be used. The method for separating may be, for example, a method using a biomolecule specifically present in the cells other than the tumor cell. Specifically, for example, the cells other than the tumor cell are bound to a ligand that specifically binds to the biomolecule via the biomolecule and the ligand to which the cells other than the tumor cell have bound is collected. The biomolecule may be, for example, a protein such as vimentin and fibronectin. The ligand may be, for example, an antibody against the protein.

A method for collecting the ligand is not particularly limited either in the method for separating the tumor cell or the method for separating the cell other than the tumor cell. For example, the ligand may be collected by binding the ligand to an affinity support that specifically binds to the ligand or, in the case where the ligand is bound to a magnetic bead, the magnetic bead may be collected by an action of magnetism.

From the viewpoint of operability, the separation step preferably includes binding the tumor cell to a magnetic bead and separating, from cells other than the tumor cell by an action of magnetism, the magnetic bead to which the tumor cell has bound, and the magnetic bead has a ligand which specifically binds to the biomolecule specifically present in the tumor cell. The biomolecule and the ligand are not particularly limited. Preferably, the biomolecule is at least one selected from the group consisting of cytokeratin and gene products of the above-described genes and the ligand is an antibody against the biomolecule. More preferably, the biomolecule is cytokeratin and the ligand is an anti-cytokeratin antibody. Specifically, commercially available products such as Anti-Cytokeratin MicroBeads (Miltenyi Biotec) may be used as the magnetic bead.

### [Collection Step]

In a collection step, a nucleic acid molecule is collected from the tumor fraction. A method for collecting a nucleic acid molecule is not particularly limited and known methods may be used. The nucleic acid molecule is not particularly limited. Examples thereof include DNA and RNA, with DNA being preferred from the viewpoint of operability.

### [Sequencing Step]

In a sequencing step, the nucleic acid molecule is subjected to sequencing. The sequencing is not particularly limited and may be, for example, NGS. An NGS method is not particularly limited and known methods may be used.

### <Method for Distinguishing Between Somatic Mutation and Germline Mutation>

A method for distinguishing between a somatic mutation and a germline mutation according to the present invention includes
the dissociating, the separating, the collecting, and the sequencing in the method for detecting a gene alteration according to the present invention, and
further includes
secondarily collecting a nucleic acid molecule from a residual fraction remaining after obtaining the tumor fraction in the separating;
secondarily sequencing the nucleic acid molecule collected in the secondarily collecting; and
estimating, for a target mutation detected in the sequencing, whether the target mutation is a germline mutation or not based on at least one of a variant allele frequency obtained in the sequencing and a variant allele frequency obtained in the secondarily sequencing. This method enables discrimination between a somatic mutation and a germline mutation without a blood sample.

### [Second Collection Step]

In a second collection step, a nucleic acid molecule is collected from a residual fraction remaining after obtaining the tumor fraction in the separation step. Details of the second collection step are the same as those of the collection step in the method for detecting a gene alteration according to the present invention.

### [Second Sequencing Step]

In a second sequencing step, the nucleic acid molecule collected in the second collection step is subjected to sequencing. Details of the second sequencing step are the same as those of the sequencing step in the method for detecting a gene alteration according to the present invention.

### [Estimation Step]

In an estimation step, for a target mutation detected in the sequencing, whether the target mutation is a germline mutation or not is estimated based on at least one of a variant allele frequency obtained in the sequencing and a variant allele frequency obtained in the secondarily sequencing. Specifically, the estimation step may be performed as described in Embodiments 1 to 3 below.

### Embodiment 1

In Embodiment 1, the estimation step includes, for a target mutation detected in the sequencing, estimating that the target mutation is a germline mutation when a VAF ratio, a ratio of a variant allele frequency obtained in the sequencing to a variant allele frequency obtained in the secondarily sequencing, is lower than a threshold. Note that, the VAF ratio corresponds to a value represented by (Variant allele frequency in tumor fraction)/(Variant allele frequency in residual fraction).

The above-described threshold in Embodiment 1 may be, for example, determined by previously analyzing a relationship between the VAF ratio and a type of mutation (somatic or germline mutation) for each population. Specifically, for example, the above-described threshold can be determined as described below. First, an FFPE tissue section and peripheral blood are collected from the same patient, a gene alteration is detected by the method for detecting a gene alteration according to the present invention, and a variant allele frequency is obtained for each of a tumor fraction and a residual fraction. On the other hand, the above-described peripheral blood is subjected to whole-exome sequencing to thereby determine whether the above-described gene alteration is a somatic mutation or a germline mutation. Based on these results, for the VAF ratio and the type of mutation, the threshold value can be determined by creating a curve used as an evaluation index in binary classification, such as a receiver operating characteristic (ROC) curve or a precision-recall (PR) curve, assuming that the above-described gene alteration is a somatic mutation.

### Embodiment 2

In Embodiment 2, the estimation step includes, for a target mutation detected in the sequencing, estimating that the target mutation is a germline mutation when a VAF difference, an absolute value of a difference between a variant allele frequency obtained in the sequencing and a variant allele frequency obtained in the secondarily sequencing, is lower than a threshold. Note that, the VAF difference corresponds to a difference represented by |(Variant allele frequency in tumor fraction)-(Variant allele frequency in residual fraction)|. The above-described threshold in Embodiment 2 may be determined in the same manner as for the above-described threshold in Embodiment 1, except that the VAF difference is used in place of the VAF ratio.

### Embodiment 3

In Embodiment 3, the estimation step includes, for a target mutation detected in the sequencing, estimating that the target mutation is a germline mutation when a variant allele frequency obtained in the secondarily sequencing is higher than a threshold. Note that, the variant allele frequency obtained in the secondarily sequencing corresponds to a variant allele frequency in the residual fraction. The above-described threshold in Embodiment 3 may be determined in the same manner as for the above-described threshold in Embodiment 1, except that the variant allele frequency obtained in the secondarily sequencing is used in place of the VAF ratio.

### EXAMPLES

Hereinafter, the present invention will be described more specifically by illustrating Examples, but the scope of the present invention is not limited to these Examples.

### Experimental Method

### [Clinical Samples]

Two diffuse-type and two intestinal gastric cancers were extracted from the Japanese pan-cancer cohort (project HOPE) including 5,521 tumor specimens. These samples were clinicopathologically diagnosed by a pathologist after surgery. Tumors were dissected from surgical specimens immediately after resection of the lesion at the Shizuoka Cancer Center Hospital, and then the specimens were stored as FFPE tissues. In addition, peripheral blood was collected as a paired control to exclude germline mutations. Details of experimental protocols have been previously described (Nagashima, T. et al. Cancer Sci 111, 687-699 (2020); Hatakeyama, K. et al. Cancer Sci 110, 2620-2628 (2019); Nagashima, T. et al. Biomed Res 37, 359-366 (2016); Shimoda, Y. et al. Biomed Res 37, 367-379 (2016); Urakami, K. et al. Biomed Res 37, 51-62, (2016); Ohshima, K. et al. Sci Rep 7, 641 (2017)). Briefly, DNA was extracted from tissues and peripheral blood samples using a QIAamp DNA Blood Mini Kit (Qiagen, Venlo, The Netherlands). The resulting DNA was purified and quantified using a NanoDrop and a Qubit 2.0 Fluorometer (Thermo Fisher Scientific, Waltham, MA).

### [Dissociation and Suspension of FFPE Tissue Samples]

FFPE tissue blocks of the gastric cancers were cut into 10, 20, and 50 μm thick sections. These sections were dewaxed by 10 min incubation in xylene thrice and then rehydrated by 30 s incubation sequentially in each of the following dilutions of ethanol: 100% (two times), 70%, 50%, and 30%. The above-described hydration process was completed with 30 s incubations in deionized water. The thus-dewaxed samples were suspended using a gentleMACS Octo Dissociator with Heaters (Miltenyi Biotec, Bergisch Gladbach, Germany), after heat-induced antigen retrieval was performed according to the manufacturer's protocol.

### [Isolation and Staining of Cells]

Fully automated cell labeling and separation were performed using an autoMACS Pro Separator (Miltenyi Biotec) according to the manufacturer's protocol. Specifically, cell suspensions derived from the FFPE tissue sections were separated using an Anti-Cytokeratin MicroBeads (Miltenyi Biotec). Cells in the resulting cell suspensions were stained using anti-cytokeratin-FITC (clone REA831, Miltenyi Biotec), anti-vimentin-APC (clone REA409, Miltenyi Biotec), and CD235a (Glycophorin A)-PE (clone REA175, Miltenyi Biotec) antibodies. Nuclei were stained with a DAPI Staining Solution (Miltenyi Biotec).

### [DNA Isolation]

DNA was extracted from the FFPE tissue and peripheral blood samples using a GeneRead DNA FFPE Kit and a QIAamp DNA blood Mini Kit (Qiagen), respectively. The resulting DNA was purified and quantified using a NanoDrop and a Qubit 2.0 Fluorometer (Thermo Fisher Scientific). To check the quality of the DNA, DIN was determined using a TapeStation (Agilent Technologies, Santa Clara, CA).

### [Targeted Sequencing of Gene Panel]

For targeted sequencing genes in DNA isolated from the FFPE tissue, a library consisting of 225 genes (listed in Table 1) was constructed using a hybridization-based enrichment protocol (SureSelect Custom panel, Agilent). In total, 2.427 Mb of the human genome, including 0.723 Mb exon regions of a RefSeq gene, were covered by 55,765 biotinylated RNA oligomers (each 120 bp in length). Binary raw data derived from a sequencer were converted into sequence reads using a bc12fastq (ver. 2.20, Illumina) that were mapped to the reference human genome (UCSC hg19). To reduce false-positive findings, mutations fulfilling any of the following criteria were eliminated: (1) a quality score <20; (2) a depth of coverage<100; (3) a depth of coverage for the alternate allele<5; (4) VAF<0.5%; and (5) not fitting filtering criteria of a variant caller (a FILTER field of a VCF record was not “PASS”). After annotating the mutations, those with an allele frequency of 1% or more in any of the below-described databases were excluded as common SNPs: (1) the 1000 genomes project (global or East Asia); (2) ExAC; and (3) gnomAD. In addition, mutations that appeared to affect protein structure, namely, missense variants, splice acceptor variants, splice donor variants, splice region variants, stop-gain variants, stop-lost variants, stop-retained variants, 5′-untranslated region premature start codon gain variants, exon-loss variants, disruptive inframe deletions, disruptive inframe insertions, frameshift variants, inframe deletions, inframe insertions, or initiator codon variants were extracted. To ensure reproducibility of the sequencing, mutations with VAF 3% were defined as valid mutations. A tumor content was estimated by an All-FIT algorithm based on tumor-only sequencing data (Loh, J. W. et al. Bioinformatics 36, 2173-2180, (2020)).

TABLE 1
Target gene (225 genes)

| | | | | | | | |
|---|---|---|---|---|---|---|---|
| ABL1 | CCND1 | ENG | IDH1 | MITF | PDGFRA | SDHAF2 | TSC1 |
| ACTN4 | CD274 | ENO1 | IGF1R | MKRN1 | PDGFRB | SDHB | TSC2 |
| ACVR1B | CD74 | EP300 | IGF2 | MLH1 | PHOX2B | SDHC | TSHR |
| AKT1 | CDC73 | EPAS1 | IL7R | MSH2 | PIK3CA | SDHD | U2AF1 |
| AKT2 | CDH1 | ERBB2 | IRF4 | MSH6 | PIK3R1 | SETD2 | UGT1A1 |
| AKT3 | CDK4 | ERBB3 | JAK1 | MTOR | PIK3R2 | SF3B1 | VHL |
| ALK | CDK6 | ERBB4 | JAK2 | MUTYH | PMS2 | SH2D1A | VTI1A |
| AMER1 | CDKN1A | ERG | JAK3 | MYB | POLD1 | SKP2 | WT1 |
| APC | CDKN1B | ESR1 | JUN | MYC | POLE | SMAD2 | |
| AR | CDKN2A | EXT1 | KDM5C | MYCL | PPP2R1A | SMAD4 | |
| ARAF | CDKN2B | EXT2 | KDM6A | MYCN | PRDM1 | SMARCA4 | |
| ARID1A | CDKN2C | EZH2 | KEAP1 | MYD88 | PRKAR1A | SMARCB1 | |
| ARID1B | CHEK2 | EZR | KIAA1549 | NCOA3 | PRKCI | SMO | |
| ARID2 | CIC | FANCC | KIF1B | NCOA4 | PTCH1 | SOX2 | |
| ATM | COL1A1 | FAT1 | KIF5B | NCOR1 | PTEN | SOX9 | |
| ATRX | CREBBP | FBXW7 | KIT | NF1 | PTPRK | SPOP | |
| AXIN1 | CRKL | FGFR1 | KLF4 | NF2 | RAC1 | STAG2 | |
| AXL | CRLF2 | FGFR2 | KMT2C | NFE2L2 | RAC2 | STAT3 | |
| B2M | CSF1R | FGFR3 | KRAS | NFIB | RAD51C | STK11 | |
| BAP1 | CTCF | FGFR4 | LMO1 | NKX2-1 | RAF1 | STRN | |
| BARD1 | CTLA4 | FH | MAP2K1 | NOTCH1 | RB1 | TACC3 | |
| BAX | CTNNB1 | FLCN | MAP2K4 | NOTCH2 | RECQL4 | TCF7L2 | |
| BCL10 | CUL3 | FOXL2 | MAP3K1 | NOTCH3 | RET | TEK | |
| BCL2L11 | CYLD | FUBP1 | MAP3K4 | NRAS | RHOA | TERT | |
| BMPR1A | DAXX | G6PD | MAPK1 | NRG1 | RNF43 | TMEM127 | |
| BRAF | DDR2 | GATA3 | MAX | NTRK1 | ROS1 | TMPRSS2 | |
| BRCA1 | DNMT1 | GNA11 | MDM2 | NTRK2 | RRAS2 | TP53 | |
| BRCA2 | DPYD | GNAQ | MDM4 | NTRK3 | RSPO2 | TP63 | |
| CARD11 | EGFR | GNAS | MED12 | PALB2 | RSPO3 | TPM3 | |
| CASP8 | EIF3E | HNF1A | MEN1 | PBRM1 | SALL4 | TPMT | |
| CCDC6 | EML4 | HRAS | MET | PDGFB | SDC4 | TRAF7 | |

### [Whole-Exome Sequencing]

To accurately distinguish germline mutations without an estimation based on databases, a pipeline described in the article (Nagashima, T. et al. Cancer Sci 111, 687-699 (2020)) was used. In brief, an exome library was constructed using an Ion Torrent AmpliSeq RDY Exome Kit (Thermo Fisher Scientific). The exome library supplied 292,903 amplicons covering 57.7 Mb of the human genome, including 34.8 Mb of exon sequences from 18,835 genes registered in the Ref-Seq. To avoid sequencer—and amplicon-derived errors, arbitrary somatic mutations were manually inspected using an Integrative Genomics Viewer (IGV), and somatic mutation candidates containing multiple nucleotide variations (about 1000 sites) were validated by Sanger sequencing.

### [Statistical Analysis]

A significant difference in read depth and VAF (including VAF ratio) was determined using a Welch's t-test. Bonferroni correction was performed for multiple comparisons. A P-value<0.01 was considered significant.

[Extraction of Gene Capable of being Used for Separating Cell]

In the above-described separation of cells, cytokeratin was used as a biomolecule specifically present in a tumor cell and an anti-cytokeratin antibody was used as a ligand which specifically bound to the biomolecule. In order to identify the biomolecule other than cytokeratin, genes expressing without being affected by tumor heterogeneity were extracted by a gene expression analysis. Note that, candidate genes desirably do not express in a normal site (non-tumor site).

Specific extraction method is as described below. In order to extract genes expressing across cancer types, 21 tumor types that the applicant had their expression information in both tumor and non-tumor sites were selected from tumors classified based on OncoTree (Kundra et al., JCO Clinical Cancer Informatics 2021).

From gene probes on a DNA microarray (Agilent Technologies), 20,869 genes coding for proteins were selected. At that time, genes coding for hypothetical proteins, genes coding for putative proteins, and probes for lincRNA detection were excluded. The DNA microarrays were used to detect expression levels in the tumor and non-tumor sites of the above-described 21 tumor types, and genes for which an average value of (Expression level in tumor site)/(Expression level in non-tumor site) was 2 or more in 95% or more of the tumor types, that is, in 20 of the above-described 21 tumor types or in all 21 tumor types were extracted from the above-described 20,869 genes.

### Experimental Results

### [Tumor Cell Enrichment Using Tissue Suspension]

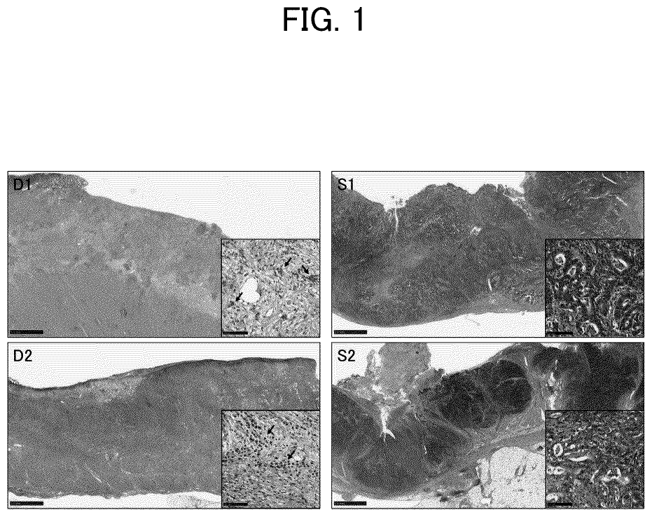

A total of 12 FFPE samples from 4 patients with gastric cancer were obtained from the tissue bank of Division of Pathology at Shizuoka Cancer Center. The samples included 10, 20, and 50 μm thick FFPE tissue sections from two diffuse-type (D1 and D2) and two intestinal (S1 and S2) gastric cancers that were collected between 2014 and 2019 (FIG. 1). A tumor cellularity, i.e., a proportion of tumor cells in the FFPE tissue sections estimated by a pathologist was less in the diffuse-type (D1, 20%; D2, 20%) than in the intestinal type (S1, 60%; S2, 50%). These diffuse-type gastric cancers were considered unsuitable for macrodissection to enrich tumor cells in the FFPE tissue sections.

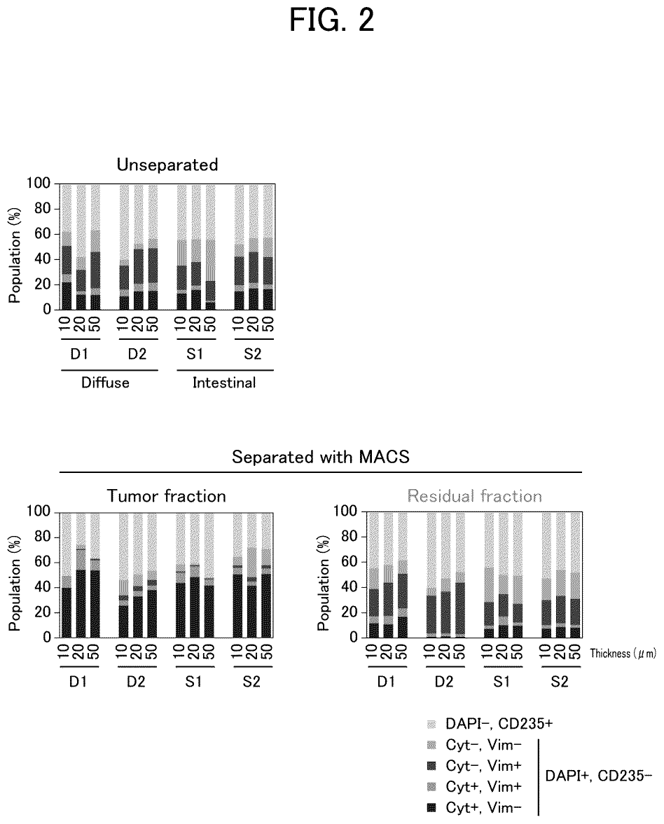

To increase the proportion of tumor cells from which DNA could be extracted in the FFPE tissue sections, tumor cell enrichment was performed using tissue suspension. As a result, cell populations considered to be of tumor cells (cytokeratin+, vimentin−) were enriched in a tumor fraction compared to unseparated samples, whereas in a residual fraction, these cell populations were decreased in both diffuse-type and intestinal gastric cancers (FIG. 2). Furthermore, no difference in the enrichment because of the thickness of the FFPE tissue sections was observed. These results indicate that tumor cells expressing cytokeratin on their surfaces could be enriched from the FFPE tissue sections of gastric cancer with low tumor content.

### [Confirmation of Sample Quality for Sequencing]

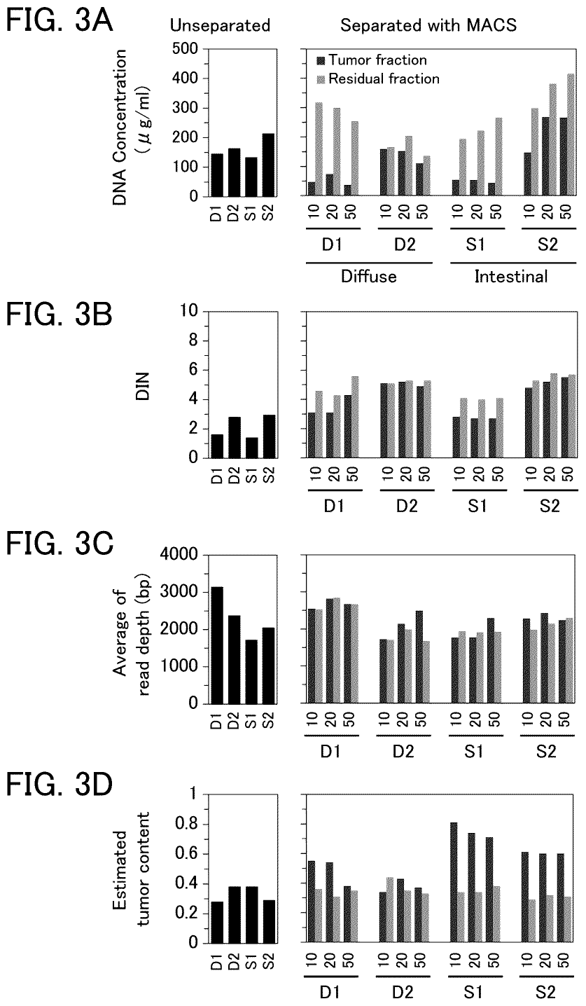

We investigated suitability of quality of DNA extracted from tissue suspension samples for NGS. Based on indicators of DNA degradation, DNA integrity number (DIN), and DNA concentration, the quality of DNA was deemed suitable for NGS (FIGS. 3A and 3B). These samples were used for library construction and NGS. Read depth of the unseparated and separated fractions was similar (FIG. 3C). Based on NGS, the tumor content was found to be increased in most of the samples in the tumor fractions (FIG. 3D). These results suggest that NGS was properly performed for the tumor fractions from the tissue suspension samples. Furthermore, although 50 μm-thick sections are recommended for preparation of the tissue suspensions, read quality of the NGS was not affected by the thickness of the FFPE tissue sections. Therefore, we concluded that NGS could be performed by tissue suspension using 10 μm-thick FFPE tissue sections. Subsequent experiments were carried out with the 10 μm-thick sections.

### [Effect of Tumor Cell Enrichment]

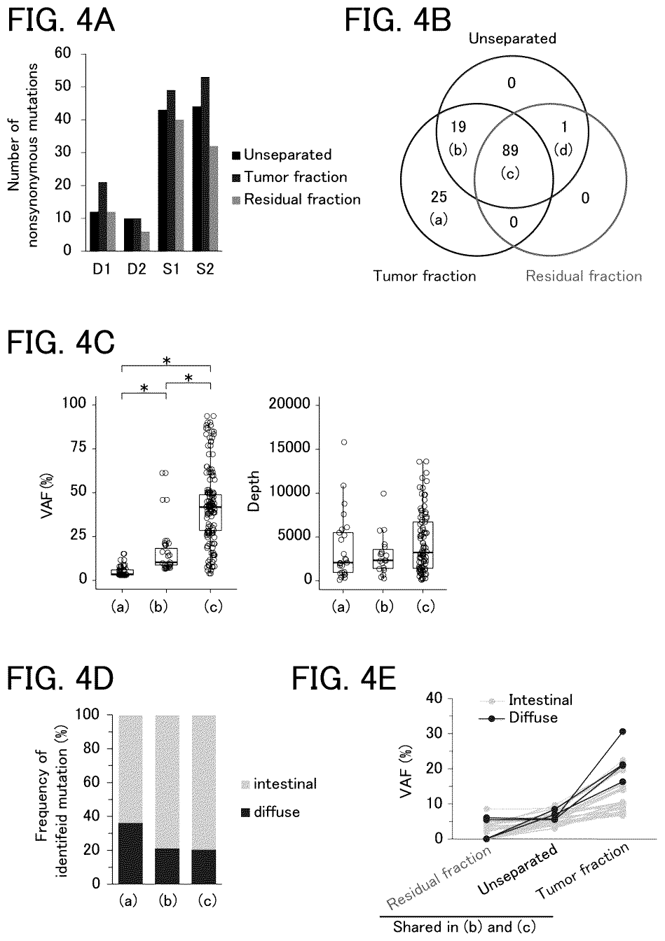

To investigate whether tumor cell enrichment using the tissue suspension affects detection of somatic mutations, we identified nonsynonymous mutations using targeted sequencing of a panel of genes (225 genes listed in Table 1 were targeted). The number of mutations detected in the tumor fraction was equal to or greater than that detected in the unseparated sample, whereas fewer mutations than that detected in the unseparated sample were detected in the residual fraction (FIG. 4A). Furthermore, 19% (25/133) of the mutations detected in the tumor fractions were tumor fraction specific (FIG. 4B). These specific mutations (a) had a significantly lower variant allele frequency (VAF) than the mutations in (b) and (c) (see FIG. 4B) for mutations (a), (b), (c), and (d)), although there was no difference in the read depth (FIG. 4C). These results suggest that tumor cell enrichment using the tissue suspension aids in identification of somatic mutations that are undetected by conventional methods. Interestingly, the tumor fraction-specific mutations (a) accounted for more than 30% of the mutations found in diffuse gastric cancer, suggesting that the tumor cell enrichment according to the present invention contributes to better detection of mutations in this cancer type with low tumor content (FIG. 4D). For mutations that were common between the tumor fraction and unseparated samples, the VAF was increased upon tumor cell enrichment (FIG. 4E).

### [Estimation of Germline Mutations Based on Differences Between Tumor and Residual Fractions]

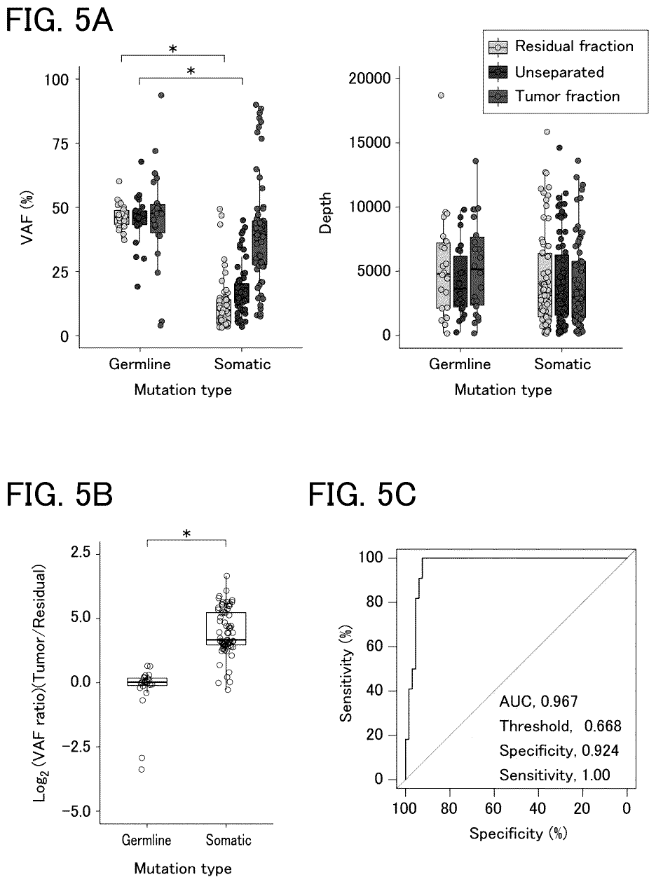

Mutations detected in sequencing of the target panel of genes excluded germline mutations present in multiple databases. Therefore, SNPs that are not registered in the databases, including those related to population differences, are identified as somatic mutations. To accurately discriminate such mutations between germline and somatic mutations, we performed whole-exome sequencing (WES) of peripheral blood from the patient who donated a tumor tissue. In target panel sequencing, 24 (18%) mutations were found as germline mutations (Tables 2-1 to 2-3). A VAF of somatic mutations found from the WES on the peripheral blood was significantly decreased in the unseparated sample and residual fraction, although there was no difference in the read depth (FIG. 5A). Additionally, germline mutations found from the WES on the peripheral blood contained one mutation shared in the unseparated sample and residual fraction ((d) in FIG. 4B). This result raises the possibility that the VAF of the germline mutations found from the WES on the peripheral blood is independent of the tumor content in FFPE tissue sections. Based on this hypothesis, the VAF ratio of the shared mutations ((c) in FIG. 4B) was compared between the germline and somatic mutations found from the WES on the peripheral blood. This ratio was significantly increased with true somatic mutations (FIG. 5B). Furthermore, a receiver operating characteristic (ROC) curve was generated to distinguish between somatic and germline mutations using the VAF ratios. An area under the curve (AUC) was 0.967 with the VAF ratio of 0.668 as the threshold (FIG. 5C). These results indicate that the VAF ratio using the tumor and residual fractions derived from FFPE tissue sections enables the estimation of germline mutations.

TABLE 2-1

| | | VAF | | | depth | | | |
|---|---|---|---|---|---|---|---|---|
| | | | Un- | | | Un- | | Discrimination |
| Symbol_positionRef > Var | sample | Tumor | separated | Residual | Tumor | separated | Residual | using blood |
| CDH1_c.1321-1G > T | D1 | 79.16 | 11.65 | 9.39 | 1243 | 1872 | 2555 | somatic |
| RECQL4_c.1064G > A | D1 | 59.79 | 46.39 | 49.07 | 9828 | 1595 | 9238 | germline |
| TCF7L2_c.1593G > T | D1 | 57.4 | 44.95 | 49.37 | 6453 | 10068 | 6121 | somatic |
| PDGFRB_c.2258C > T | D1 | 51.56 | 53.1 | 50.09 | 7244 | 2030 | 6903 | germline |
| PDGFRB_c.2972G > A | D1 | 50.28 | 50.12 | 50.96 | 9234 | 2594 | 9605 | germline |
| BRCA1_c.2726A > T | D1 | 48.81 | 42.76 | 42.95 | 1172 | 4090 | 1411 | germline |
| POLD1_c.512C > T | D1 | 48.4 | 45.7 | 48.03 | 13586 | 9786 | 18716 | germline |
| TSC2_c.3475C > T | D1 | 43.15 | 36.26 | 45.33 | 4857 | 2780 | 7317 | germline |
| ATRX_c.1492A > G | D1 | 42.44 | 47.07 | 46.3 | 1593 | 4738 | 1177 | germline |
| MTOR_c.61G > A | D1 | 39.27 | 12.67 | 10.05 | 5113 | 3251 | 6369 | somatic |
| BRAF_c.1406G > T | D1 | 30.6 | 5.59 | 6.03 | 1585 | 7502 | 1874 | somatic |
| JAK2_c.3144C > A | D1 | 20.91 | 5.47 | 5.44 | 1368 | 4640 | 1158 | somatic |
| NOTCH3_c.4039G > C | D2 | 71.97 | 48.75 | 45.83 | 157 | 240 | 144 | somatic |
| RECQL4_c.1321C > T | D2 | 47.39 | 45.84 | 47.03 | 9908 | 9208 | 5575 | germline |
| PDGFB_c.35G > T | D2 | 43.96 | 40.6 | 43.31 | 2134 | 2473 | 1905 | somatic |
| STK11_c.437A > G | D2 | 38.59 | 39.81 | 46.79 | 3239 | 4105 | 3552 | somatic |
| PHOX2B_c.765_779delGGCAGCGGCGGCAGC | D2 | 24.51 | 19.13 | 39.59 | 971 | 1286 | 821 | somatic |
| NOTCH2_c.7_8delinsTT | D2 | 11.78 | 10.27 | 8.94 | 2970 | 3632 | 3108 | somatic |
| NOTCH3_c.3523C > T | S1 | 93.66 | 67.81 | 60.18 | 2966 | 4001 | 4422 | germline |
| SMARCA4_c.2092G > A | S1 | 88.43 | 37.25 | 29.75 | 3120 | 3313 | 3526 | somatic |
| RNF43_c.575delC | S1 | 84.83 | 35.46 | 27.48 | 2940 | 3663 | 4159 | somatic |
| BAX_c.121delG | S1 | 81.36 | 34.33 | 24.59 | 7638 | 9338 | 10916 | somatic |
| MAP2K1_c.371C > T | S1 | 64.83 | 26.83 | 17.06 | 2249 | 2169 | 2679 | somatic |
| KIAA1549_c.5191G > C | S1 | 61.37 | 53.9 | 53.11 | 9811 | 8606 | 9487 | germline |
| PIK3CA_c.3140A > G | S1 | 50.13 | 24.1 | 21.35 | 1137 | 697 | 726 | somatic |
| PTCH1_c.3907C > T | S1 | 49.66 | 46.48 | 44.61 | 6053 | 6659 | 6792 | germline |
| TACC3_c.2227G > A | S1 | 47.81 | 48.43 | 46.02 | 2675 | 3285 | 3553 | germline |
| PTCH1_c.3606delC | S1 | 44.81 | 22.46 | 16.71 | 10588 | 9652 | 11110 | somatic |
| TEK_c.1250delC | S1 | 44.69 | 21.81 | 17.75 | 1289 | 1073 | 1234 | somatic |
| TMPRSS2_c.137C > T | S1 | 44.01 | 18.93 | 14.82 | 8248 | 8068 | 9194 | somatic |
| TSC2_c.2072G > A | S1 | 43.54 | 20.14 | 14.19 | 1525 | 1822 | 2170 | somatic |
| CASP8_c.1177A > G | S1 | 42.64 | 19.39 | 14.1 | 2031 | 1604 | 2007 | somatic |
| CTNNB1_c.1346G > A | S1 | 42.46 | 18.05 | 14.1 | 3375 | 3041 | 3411 | somatic |
| ERBB3_c.1442G > A | S1 | 42.45 | 19.34 | 14.36 | 2641 | 2720 | 3072 | somatic |
| MSH6_c.407A > T | S1 | 41.82 | 18.88 | 13.21 | 1363 | 1372 | 1476 | somatic |
| FAT1_c.12629A > T | S1 | 41.71 | 21.62 | 14.79 | 2201 | 2077 | 2136 | somatic |
| JAK1_c.425dupA | S1 | 40.37 | 16.94 | 14.14 | 2695 | 2656 | 3105 | somatic |
| TP53_c.91G > A | S1 | 39.29 | 16.38 | 13.83 | 761 | 995 | 1077 | somatic |
| FAT1_c.3423G > C | S1 | 39.27 | 43.36 | 37.25 | 1416 | 1100 | 1345 | germline |
| ARID1A_c.2382dupG | S1 | 38.86 | 15.64 | 13.52 | 2831 | 2488 | 2862 | somatic |
| ATM_c.1010G > A | S1 | 38.6 | 13.5 | 13.71 | 285 | 274 | 350 | somatic |
| ARID1A_c.5548dupG | S1 | 38.23 | 17 | 12.03 | 5087 | 5870 | 6448 | somatic |
| FLCN_c.1285delC | S1 | 38 | 19.26 | 13.13 | 7137 | 8074 | 9138 | somatic |
| NOTCH1_c.5950C > T | S1 | 37.38 | 17.72 | 13.68 | 11739 | 14612 | 15867 | somatic |
| SMARCB1_c.1091_1093delAGA | S1 | 36.5 | 17.15 | 11.87 | 5737 | 6863 | 7091 | somatic |
| AXIN1_c.1523delG | S1 | 35.65 | 16.83 | 13.52 | 8489 | 10713 | 12664 | somatic |
| SALL4_c.3149T > C | S1 | 31.96 | 43.34 | 42 | 2638 | 2118 | 2150 | germline |
| BRAF_c.1447A > G | S1 | 29.96 | 13.22 | 11.02 | 998 | 749 | 717 | somatic |
| SALL4_c.2983delG | S1 | 28.25 | 15.85 | 11.76 | 3759 | 3173 | 3495 | somatic |
| PIK3CA_c.323G > A | S1 | 26.97 | 14.55 | 11.57 | 660 | 440 | 432 | somatic |
| SALL4_c.200G > A | S1 | 25.83 | 12.75 | 10.39 | 4302 | 4518 | 4803 | somatic |

TABLE 2-2

| | | | | | | | | |
|---|---|---|---|---|---|---|---|---|
| FGFR3_c.2414G > A | S1 | 15.58 | 5.93 | 4.04 | 7515 | 9289 | 10098 | somatic |
| GATA3_c.708delC | S1 | 14.29 | 5.01 | 3.29 | 5801 | 6985 | 7485 | somatic |
| FH_c.956A > G | S1 | 10.53 | 7.13 | 4.03 | 874 | 743 | 917 | somatic |
| NOTCH2_c.7_8delinsTT | S1 | 8.53 | 8.76 | 8.6 | 8011 | 9393 | 10552 | somatic |
| FBXW7_c.1712G > T | S1 | 7.94 | 3.49 | 3.82 | 1411 | 1116 | 1388 | somatic |
| FGFR1_c.1052A > G | S1 | 7.49 | 6.29 | 4.66 | 2990 | 2814 | 3092 | somatic |
| MSH2_c.2131C > T | S2 | 90 | 33.6 | 10.16 | 2449 | 3057 | 2421 | somatic |
| ARAF_c.763delC | S2 | 86.78 | 42.2 | 13.99 | 3836 | 3019 | 3003 | somatic |
| B2M_c.43_44delCT | S2 | 83.34 | 30.75 | 9.51 | 7292 | 6049 | 6968 | somatic |
| ARID1A_c.2296dupC | S2 | 76.76 | 20.41 | 4.29 | 1437 | 2092 | 2567 | somatic |
| SALL4_c.1018G > A | S2 | 63.3 | 54.75 | 51.49 | 6714 | 4866 | 4700 | germline |
| BAX_c.121delG | S2 | 61.63 | 21.41 | 5.14 | 10684 | 10213 | 11553 | somatic |
| ARID2_c.5305C > T | S2 | 49.48 | 17.61 | 10.71 | 291 | 318 | 252 | somatic |
| APC_c.656C > T | S2 | 49.45 | 13.92 | 6.9 | 182 | 237 | 203 | somatic |
| PDGFRB_c.2972G > A | S2 | 47.45 | 45.59 | 46.5 | 7812 | 7014 | 7721 | germline |
| TERT_c.358C > T | S2 | 47.15 | 18.77 | 6.21 | 3334 | 2690 | 3093 | somatic |
| CDC73_c.968T > C | S2 | 45.45 | 13.73 | 5.69 | 814 | 772 | 808 | somatic |
| SDHD_c.331G > A | S2 | 45.16 | 47.54 | 47.55 | 2263 | 2503 | 2105 | germline |
| TP53_c.586C > T | S2 | 43.74 | 16.19 | 6.01 | 2835 | 2459 | 2747 | somatic |
| NOTCH1_c.1334C > T | S2 | 43.43 | 17.28 | 4.89 | 11362 | 9185 | 11422 | somatic |
| ERBB2_c.838_839delinsTT | S2 | 41.99 | 17.59 | 5.03 | 4001 | 3717 | 3939 | somatic |
| CREBBP_c.5488G > A | S2 | 41.77 | 15.87 | 4.49 | 12323 | 10252 | 12701 | somatic |
| FAT1_c.3784C > T | S2 | 39.51 | 16.92 | 3.81 | 5270 | 4847 | 4934 | somatic |
| ARID2_c.2806G > T | S2 | 38.83 | 43.23 | 44.73 | 6694 | 6591 | 5384 | germline |
| PIK3CA_c.2308C > T | S2 | 38.79 | 24.41 | 4.9 | 348 | 295 | 286 | somatic |
| ACVR1B_c.1136 + 2T > C | S2 | 31.5 | 19.06 | 6.88 | 5013 | 4507 | 4000 | somatic |
| RET_c.1942G > A | S2 | 31.04 | 12.94 | 3.18 | 13619 | 11070 | 12644 | somatic |
| SALL4_c.2996C > T | S2 | 28.49 | 13.47 | 4.36 | 5448 | 4144 | 3761 | somatic |
| EXT1_c.369delA | S2 | 28.12 | 13.69 | 4.2 | 6953 | 5071 | 4481 | somatic |
| CARD11_c.2707G > A | S2 | 27.27 | 13.14 | 4.14 | 5468 | 3951 | 4030 | somatic |
| RAF1_c.770C > T | S2 | 26.98 | 14.75 | 5.87 | 4337 | 4557 | 3953 | somatic |
| GNAS_c.2153A > T | S2 | 26.93 | 10.86 | 3.33 | 1957 | 1556 | 1411 | somatic |
| ACVR1B_c.85delG | S2 | 20.83 | 9.7 | 3.57 | 509 | 402 | 392 | somatic |
| CDH1_c.2245C > T | S2 | 19.57 | 8.64 | 4.43 | 1242 | 1319 | 1219 | somatic |
| KLF4_c.709G > A | S2 | 18.59 | 10.08 | 3.53 | 8004 | 6481 | 7261 | somatic |
| NF1_c.611T > C | S2 | 14.62 | 5.88 | 4.84 | 130 | 119 | 124 | somatic |
| ALK_c.4573A > G | S2 | 5.59 | 30.61 | 42.53 | 3705 | 3247 | 3348 | germline |
| ALK_c.1289C > A | S2 | 3.95 | 29.94 | 41.13 | 5421 | 4913 | 4872 | germline |
| TP53_c.529_546del | D2 | 61.18 | 26.03 | 2.37 | 3297 | 4936 | 4091 | somatic |
| ARID1A_c.1113dupG | D2 | 46.03 | 22.86 | 0 | 252 | 280 | NA | somatic |
| MED12_c.5429G > T | D2 | 21.14 | 8.37 | 0 | 2866 | 4016 | NA | somatic |
| KIF1B_c.4406G > A | D2 | 16.28 | 6.88 | 0 | 1241 | 1658 | NA | somatic |
| BRCA2_c.3019G > T | S1 | 13.92 | 5.38 | 0 | 431 | 260 | NA | somatic |
| KIAA 1549_c.3974G > A | S1 | 9.3 | 3.77 | 2.56 | 3872 | 3100 | 3470 | somatic |
| FAT1_c.2510T > C | S1 | 7.12 | 3.68 | 2.85 | 3116 | 2367 | 2740 | somatic |
| CDH1_c.2494G > A | S2 | 22.55 | 7.29 | 0 | 2333 | 2263 | NA | somatic |
| CD74_c.51G > A | S2 | 20.25 | 5.92 | 0 | 5738 | 4492 | NA | somatic |
| NKX2-1_c.349A > G | S2 | 15.88 | 5.9 | 0 | 2292 | 1798 | NA | somatic |
| PIK3CA_c.3140A > G | S2 | 14.39 | 6.08 | 0 | 660 | 724 | NA | somatic |
| MAP3K4_c.866A > G | S2 | 10.25 | 6.53 | 2.01 | 2058 | 2525 | 1994 | somatic |
| JAK1_c.2580delA | S2 | 10.16 | 4.51 | 0 | 3023 | 2597 | NA | somatic |
| AXL_c.379G > A | S2 | 9.85 | 4.64 | 0 | 5819 | 5777 | NA | somatic |
| SMO_c.1199G > A | S2 | 9.09 | 3.01 | 0 | 9964 | 7216 | NA | somatic |
| FAT1_c.8965delA | S2 | 8.5 | 5.57 | 2.61 | 1471 | 1347 | 1377 | somatic |
| DAXX_c.1884dupC | S2 | 7.85 | 3.91 | 0 | 1363 | 1354 | NA | somatic |

TABLE 2-3

| | | | | | | | | |
|---|---|---|---|---|---|---|---|---|
| ACTN4_c.409G > A | S2 | 7.15 | 4.83 | 0 | 4168 | 3540 | NA | somatic |
| ROS1_c.1679G > A | S2 | 6.69 | 4.46 | 0 | 2273 | 2083 | NA | somatic |
| PTEN_c.968dupA | D1 | 8.13 | 0 | 0 | 123 | NA | NA | germline |
| PTEN_c.532_534delTAT | D1 | 6.79 | 0 | 0 | 854 | NA | NA | somatic |
| HNF1A_c.872delC | D1 | 3.47 | 0 | 0 | 5854 | NA | NA | somatic |
| ACVR1B_c.1261 + 2T > G | D1 | 3.23 | 0 | 0 | 1983 | NA | NA | somatic |
| AXIN1_c.1597C > T | D1 | 3.09 | 0 | 0 | 15813 | NA | NA | somatic |
| ERBB4_c.3641A > G | D1 | 3.09 | 0 | 0 | 6109 | NA | NA | somatic |
| ACVR1B_c.652T > C | D1 | 3.02 | 0 | 0 | 5200 | NA | NA | somatic |
| EZR_c.-122G > T | D1 | 3.02 | 0 | 0 | 5500 | NA | NA | somatic |
| EPAS1_c.955C > A | S1 | 5.22 | 0 | 0 | 7599 | NA | NA | somatic |
| CYLD_c.88G > A | S1 | 4.39 | 0 | 0 | 683 | NA | NA | somatic |
| AXIN1_c.1333C > T | S1 | 3.58 | 0 | 0 | 10850 | NA | NA | somatic |
| BRCA2_c.2957delA | S1 | 3.49 | 0 | 0 | 344 | NA | NA | somatic |
| TEK_c.255delA | S1 | 3.1 | 0 | 0 | 1744 | NA | NA | somatic |
| EPAS1_c.1658C > T | S1 | 3.09 | 0 | 0 | 4692 | NA | NA | somatic |
| SOX2_c.229G > A | S1 | 3.07 | 0 | 0 | 8784 | NA | NA | somatic |
| PALB2_c.1675_1676delinsTG | S2 | 6.02 | 0 | 0 | 980 | NA | NA | somatic |
| CRKL_c.491G > A | S2 | 5.49 | 0 | 0 | 2077 | NA | NA | somatic |
| CREBBP_c.3250delA | S2 | 3.53 | 0 | 0 | 2494 | NA | NA | somatic |
| SMARCA4_c.4210G > A | D1 | 3.55 | 0 | 2.55 | 1716 | NA | 2278 | somatic |
| TSHR_c.457T > A | S2 | 15.21 | 2.97 | 0 | 743 | 809 | NA | somatic |
| PRKCI_c.826delA | S2 | 11.6 | 2.78 | 0 | 957 | 899 | NA | somatic |
| CSF1R_c.1497A > G | S2 | 8.76 | 2.89 | 0 | 2055 | 1659 | NA | somatic |
| ARID1A_c.4892A > C | S2 | 7.83 | 2.89 | 0 | 3077 | 2837 | NA | somatic |
| ROS1_c.4142-1G > A | S2 | 5.71 | 2.86 | 0 | 403 | 420 | NA | somatic |
| ESR1_c.539A > G | S2 | 5.47 | 2.74 | 0 | 2415 | 3061 | NA | somatic |
| AXL_c.1503dupC | S1 | 2.42 | 22.41 | 25.11 | 2516 | 2566 | 3082 | germline |

### Conclusion

Example demonstrates that the number of detectable gene alterations and the VAF were increased. Furthermore, mutation analysis of DNA isolated from the tumor and residue fractions enabled estimation of germline mutations without a blood sample, i.e., without blood as a reference. This approach of tumor cell enrichment can not only enhance a success rate of the target panel sequencing, but also improve accuracy of detection of somatic mutations in specimens stored without blood samples, for example, as FFPE tissue sections.

[Extraction of Gene Capable of being Used for Separating Cell]

The following 46 genes were extracted from the above-described 20,869 genes:

a HJURP gene, a KIF2C gene, a ASPN gene, a GINS1 gene, a NUSAP1 gene, a IQGAP3 gene, a CDK1 gene, a TPX2 gene, a CDT1 gene, a MMP11 gene, a MEX3A gene, a TUBB3 gene, a BIRC5 gene, a HIST2H3A gene, a CENPF gene, a CCNB2 gene, a TROAP gene, a CDCA5 gene, a KIAA0101 gene, a UBE2C gene, a AURKB gene, a CKAP2L gene, a CEP55 gene, a EXO1 gene, a KIF20A gene, a CCNA2 gene, a HIST1H2AL gene, a ANLN gene, a CENPA gene, a TTK gene, a ORC6 gene, a SHCBP1 gene, a FOXM1 gene, a MELK gene, a SPC25 gene, a TOP2A gene, a BUB1B gene, a MAD2L1 gene, a MND1 gene, a KIFC1 gene, a NUF2 gene, a GTSE1 gene, a E2F1 gene, a BUB1 gene, a DLGAP5 gene, and a KIF14 gene.

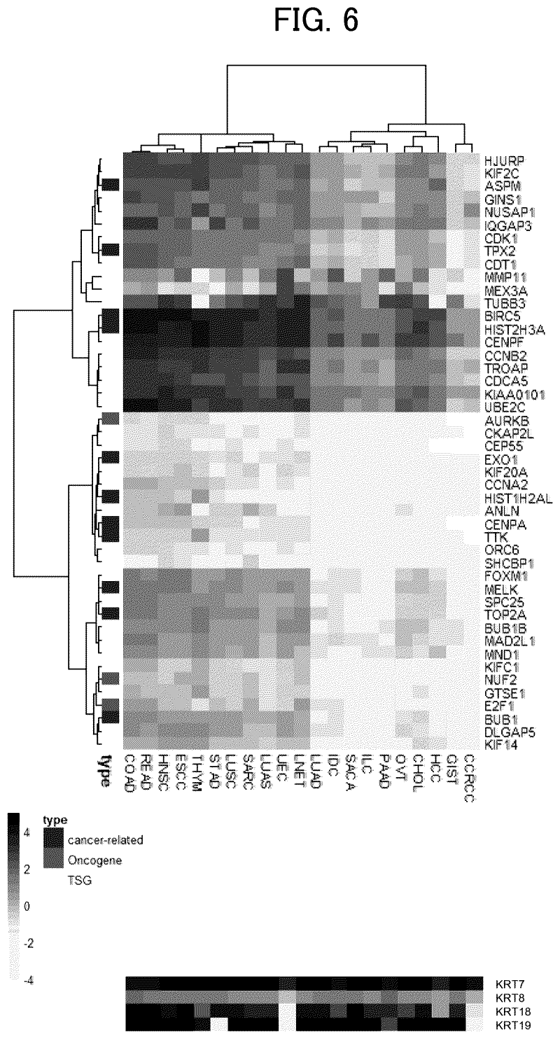

A heat map was generated by clustering expression levels in a tumor site plotted with 21 tumor types and 46 genes as axes. In FIG. 6, 46 genes from the HJURP gene to the KIF14 gene have an average value of (Expression level in tumor site)/(Expression level in non-tumor site) of 2 or more in 95% or more of 21 tumor types from CCRCC to COAD. In FIG. 6, the expression levels in the tumor site were compared among 46 genes of which expression levels in the tumor site were on average twice or more as high as those in the non-tumor site for 95% or more of the above-described tumor types. In FIG. 6, the HJURP gene to UBE2C genes tended to be relatively highly expressed in the tumor site, whereas the AURKB gene to KIF14 genes tended to be relatively poorly expressed in the tumor site. In FIG. 6, for tumors from LNET to COAD, the 46 genes tended to be relatively highly expressed in the tumor site, and for tumors from CCRCC to LUAD, the 46 genes tended to be relatively poorly expressed in the tumor site.

FIG. 6 also shows results for keratin genes (KRT7, KRT8, KRT18, and KRT19). The 46 genes tended to be less expressed in the tumor site than the keratin genes, but, for some tumors, some genes were expressed higher than the keratin genes in the tumor site.

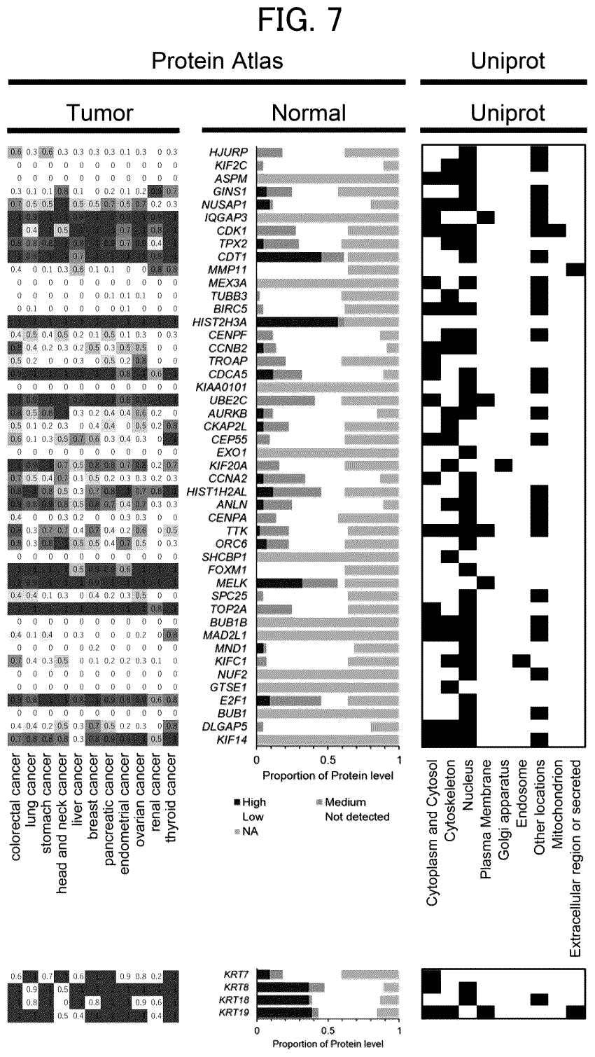

Among public databases, Protein Atlas (a database showing protein production by gene expression using immunostaining) was used to illustrate expression frequencies of the 46 genes in tumor and normal tissues, and UniProt (a database on intracellular localization of gene expression) was used to illustrate intracellular localization expression of the 46 genes (FIG. 7). FIG. 7 also shows results for the above-described keratin genes.

FIG. 7 demonstrates the following. Among the 46 genes, a plurality of genes were found to be immunostained in a tumor tissue (corresponding to the tumor site described above) to the same or greater level as keratin Among the 46 genes, a small number of genes were found to be immunostained in a normal tissue (corresponding to the non-tumor site described above) to a greater level than keratin. Therefore, the 46 genes may be used to separate tumor cells from normal cells more accurately than with the keratin. The Protein Atlas also contained some genes whose protein production could not be observed by immunostaining in the normal tissue (possibly due to antibody performance). Note that, there also were genes for which immunostaining had not been performed in the normal tissue (corresponding to the non-tumor site described above). Expression of the 46 genes tended to be localized especially in the nucleus. From the above, gene products of all 46 genes can be biomolecules to be used in the separation step.

1. A method for detecting a gene alteration, the method comprising:
dissociating a single cell population from a formalin-fixed, paraffin-embedded tissue section comprising a tumor cell;
separating a tumor fraction comprising the tumor cell from the single cell population;
collecting a nucleic acid molecule from the tumor fraction; and
sequencing the nucleic acid molecule.

2. The method for detecting a gene alteration according to claim 1, wherein the formalin-fixed, paraffin-embedded tissue section has a thickness of 10 μm or more and 50 μm or less.

3. The method for detecting a gene alteration according to claim 1, wherein the nucleic acid molecule is DNA.

4. The method for detecting a gene alteration according to claim 1, wherein the sequencing is next-generation sequencing.

5. The method for detecting a gene alteration according to claim 1, wherein the separating comprises binding the tumor cell to a magnetic bead and separating, from cells other than the tumor cell by an action of magnetism, the magnetic bead to which the tumor cell has bound,
the magnetic bead having a ligand that specifically binds to a biomolecule specifically present in the tumor cell.

6. The method for detecting a gene alteration according to claim 5, wherein the biomolecule is at least one selected from the group consisting of cytokeratin and gene products of the below-described genes and the ligand is an antibody against the biomolecule:
a HJURP gene, a KIF2C gene, a ASPN gene, a GINS1 gene, a NUSAP1 gene, a IQGAP3 gene, a CDK1 gene, a TPX2 gene, a CDT1 gene, a MMP11 gene, a MEX3A gene, a TUBB3 gene, a BIRC5 gene, a HIST2H3A gene, a CENPF gene, a CCNB2 gene, a TROAP gene, a CDCA5 gene, a KIAA0101 gene, a UBE2C gene, a AURKB gene, a CKAP2L gene, a CEP55 gene, a EXO1 gene, a KIF20A gene, a CCNA2 gene, a HISTlH2AL gene, a ANLN gene, a CENPA gene, a TTK gene, a ORC6 gene, a SHCBP1 gene, a FOXM1 gene, a MELK gene, a SPC25 gene, a TOP2A gene, a BUB1B gene, a MAD2L1 gene, a MND1 gene, a KIFC1 gene, a NUF2 gene, a GTSE1 gene, a E2F1 gene, a BUB1 gene, a DLGAP5 gene, and a KIF14 gene.

7. The method for detecting a gene alteration according to claim 5, wherein the biomolecule is cytokeratin and the ligand is an anti-cytokeratin antibody.

8. A method for distinguishing between a somatic mutation and a germline mutation, the method comprising:
the dissociating, the separating, the collecting, and the sequencing in the method for detecting a gene alteration according to claim 1, and
further comprising:
secondarily collecting a nucleic acid molecule from a residual fraction remaining after obtaining the tumor fraction in the separating;
secondarily sequencing the nucleic acid molecule collected in the secondarily collecting; and
estimating, for a target mutation detected in the sequencing, whether the target mutation is a germline mutation or not based on at least one of a variant allele frequency obtained in the sequencing and a variant allele frequency obtained in the secondarily sequencing.
